(19) United States
(12) Patent Application Publication
(10) Pub. No.: US 2023/0121718 A1
(43) Pub. Date: Apr. 20, 2023

(54) METHODS AND SYSTEMS FOR FABRICATING BIOSENSORS

(71) Applicants: California Institute of Technology, Pasadena, CA (US); Gottfried Wilhelm Leibniz Universität Hannover, Hannover (DE); Helmholtz Zentrum München - Deutsches Forschungszentrum für Gesundheit und Umwelt (GmbH), Neuherberg (DE)

(72) Inventors: Oliver PLETTENBURG, BURGDORF (DE); Christin AHLBRECHT, HANNOVER (DE); Dvin ADALIAN, LOS ANGELES, CA (US); Axel SCHERER, BARNARD, VT (US); Xiomara Linnette MADERO, GLENDALE, CA (US); Samson CHEN, PASADENA, CA (US); Muhammad Musab JILANI, PASADENA, CA (US)

(21) Appl. No.: 18/046,737

(22) Filed: Oct. 14, 2022

Related U.S. Application Data
(60) Provisional application No. 63/256,086, filed on Oct. 15, 2021.

(51) Int. Cl. G01N 27/327

(52) U.S. Cl. CPC G01N 27/3273; G01N 27/3272

(57) ABSTRACT

Methods and systems are described for fabricating thin hydrogel layers on biosensors by a drop-spin method, which includes placing a drop of the hydrogel on the electrode, spinning the wafer at high speed in a vacuum, and heating the wafer to cure. One and multilayer sensors can be fabricated in this way, by adding layers of hydrogel or metal.

### CROSS-REFERENCE TO RELATED APPLICATIONS

The present application claims priority to U.S. Provisional Patent Application No. 63/256,086 titled “Methods and Systems for Fabricating Biosensors”, filed on Oct. 15, 2021, the disclosure of which is incorporated by reference herein in its entirety.

### TECHNICAL FIELD

The present disclosure relates to the fabrication of coatings for biosensors. More particularly, it relates to the fabrication of coating biosensors with biocompatible biopolymers.

### BACKGROUND

Implantable sensors for measuring biomarkers, such as enzymatic assays, have a limited lifetime, as they are subjected to the defensive foreign body response. This response includes inflammation, recruitment of immune cells and the subsequent formation of a fibrotic capsule. This results in the loss of enzymatic activity and in turn loss of biosensing capability, as well as a dense capsule that may limit analyte diffusion, thus reducing the functional measurement window. Immune molecules also foul the electrochemical electrode surface, blocking and inactivating the electrode's redox ability. It is also the case that current enzymatic biosensor fabrication methods are difficult to scale without variability and requiring significant individual calibration.

### SUMMARY

Herein are described methods and systems to fabricate precise coatings on biosensors with biocompatible biopolymers via spincoating and curing a hydrogel. These thin, geometrically defined pullulan films have consistent pore sizing, chemical resistance, and provide a stable protective environment for enzymes.

In a first aspect of the invention, a method of fabricating a biosensor is disclosed, the method comprising: providing a wafer or other solid material with an electrode on a surface of the wafer or other solid material; placing a drop of hydrogel on the electrode; spinning the wafer, while the wafer is subjected to a partial vacuum; and heating the wafer or other solid material. Alternative to a wafer, another solid material can be used to support the electrode, such as glass, polymer, etc.

In a second aspect of the invention, a biosensor is disclosed, comprising: an electrode comprising a conductive layer over a substrate layer; and a hydrogel layer over the conductive layer, the hydrogel layer being less than 3 micrometers thick and comprising enzymes immobilized within the hydrogel layer. In some embodiments of the second aspect, the hydrogel layer is less than 1 micrometer thick. In some embodiments, the enzymes are in the oxidase family, such as glucose oxidase, lactate oxidase, uricase oxidase, alcohol oxidase, cortisol oxidase, xanthine oxidase, cholesterol oxidase, and/or sarcosine oxidase.

The details of one or more embodiments of the disclosure are set forth in the accompanying drawings and the description below. Other features, objects, and advantages will be apparent from the description, drawings, and claims.

### BRIEF DESCRIPTION OF DRAWINGS

The accompanying drawings, which are incorporated into and constitute a part of this specification, illustrate one or more embodiments of the present disclosure and, together with the description of example embodiments, serve to explain the principles and implementations of the disclosure.

FIGS. 1A and 1B show an example setup for the drop-spin process.

FIGS. 2A and 2B show an example of pullulan drop-spin coated with (FIG. 2A) and without (FIG. 2B) platinum sputtered on top.

FIGS. 3A and 3B show example scanning electron microscope (SEM) pictures of a drop-spin sensor. FIG. 3A shows an example of a sensor from the drop-coat process, and FIG. 3B shows an example of a sensor from the drop-spin process.

FIG. 4 shows examples of multilayer formed using the drop-spin process.

FIGS. 5A to 5D show examples of adhesion between the pullulan hydrogel and platinum electrode in a biosensor fabricated with drop-spin deposition.

FIGS. 6A to 6D illustrate the longevity of the drop-spin deposition in exemplary one-layer and two-layer embodiments. FIG. 6A shows an example of the one-layer embodiment. FIG. 6B shows an example of the two-layer embodiment. FIG. 6C shows an example of normalized sensitivity over time. FIG. 6D shows an example comparison of normalized (to initial measurements) sensitivity over 36 days.

FIG. 7 shows an example of concentration vs. current, for example, sensors fabricated by drop-spin.

FIG. 8 shows a further example comparison between one-layer and two-layer sensors (coated by the drop-spin fabrication method) for concentration vs. current.

FIGS. 9A and 9B show an example comparison between one-layer (FIG. 9A) and two-layer (FIG. 9B) sensors (coated by the drop-spin fabrication method) stored in phosphate-buffered saline.

FIG. 10 shows an example comparison of one-layer sensors vs. two-layer sensors (each coated by the drop-spin fabrication method) in terms of Km values over time.

FIG. 11 shows example enzyme saturation curves for sensors coated by the drop-spin fabrication method on a dry wafer over 24 days.

FIG. 12 shows an example graph of the long-term longevity of sensors coated by the drop-spin fabrication method.

FIGS. 13A-13D show an example of data for a double-layer hydrogel. FIG. 13A shows an example of the double-layer hydrogel. FIG. 13B shows an example graph of sensitivity over time. FIG. 13C shows an example graph of K values over time. FIG. 13D shows an example graph of normalized max current over time.

FIGS. 14A-14D show example scanning electron microscope (SEM) pictures of drop-coat vs. drop-spin layer thickness.

FIG. 15 shows an example of a SEM picture showing layer thickness with the drop-spin method.

FIGS. 16A-16D show examples for double-layer hydrogel sensors. The schematic composition of the sensor coating is depicted in FIG. 16A. FIG. 16B and 16C show example graphs of enzyme saturation curves for two sensors over 948 days. FIG. 16D shows an example graph of current as a function of glucose concentration on day 1148. FIG. 16E shows an example graph of enzyme saturation curve on day 1148.

FIGS. 17A-17C show example data for a double-layer coated hydrogel sensor. FIG. 17A shows an example of the double-layer hydrogel. FIG. 17B shows an example graph of current as a function of glucose concentration on day 1. FIG. 17C shows an example graph of enzyme saturation curve on day 1.

FIGS. 18A-18B show an example of data for a double-layer hydrogel. FIG. 18A shows an example of the double-layer hydrogel. FIG. 18B shows an example graph of enzyme saturation curves over 15 days.

### DETAILED DESCRIPTION

Fabricating precise coatings on biosensors with biocompatible biopolymers can be accomplished via spincoating and curing a hydrogel on the electrode. By spincoating (“drop-spin”) thin, geometrically defined films can be deposited having consistent pore sizing, chemical resistance, and be a stable protective environment for enzymes immobilized therein. An example process for immobilizing enzymes in the hydrogel can be found in EPO Application No. EP21202802.1 titled HYDROGEL FOR IMMOBILIZATION OF ONE OR MORE ENZYME(S) AND METHOD FOR PREPARING THE SAME invented by Oliver Plettenburg et. al. and filed on Oct. 15, 2021.

As used herein, an electrode is a conductive layer (such as Pt), usually on a substrate (such as Si, silicon nitride, or Si/SiO₂).

As used herein, a diffusion barrier is a layer used to protect the conducting metal layer from diffusion of impurities from another metal and from corrosion from the environment.

In some embodiments, the drop-spin process includes placing one or more spaced-apart drops of hydrogel (e.g., pullulan) on electrodes of a wafer, then spinning the wafer at high speed. The wafer can be placed under a vacuum to help hold the wafer to the spincoater platform (e.g., at 60-70 psi). The wafer can be held down with an adhesive in addition as an alternative. In some embodiments, the electrodes are platinum. In some embodiments, the electrodes are made of another noble metal (e.g., gold, silver), a platinum group metal, a biocompatible material like graphite or a metal such as titanium, or an oxide (e.g., SiO₂, titanium oxide or Al₂O₃), a non-noble metal, a layer of nanoparticles or nanotubes, or other appropriate conducting material for depositing on a sensor as known in the art. In some embodiments, the process is followed by a heating step.

Hydrogels are three-dimensional networks of hydrophilic polymers. Polysaccharide polymers can be used in all applications, but other polymers may be used when the hydrogel is being used as a second protective layer. The hydrogels can comprise one or more polysaccharides differing e.g., in carbohydrate composition or molecular weight. Furthermore, the carbohydrate polymers can be functionalized to for covalent bonds with each other or with the other species. In addition, the hydrogel can be used in combination with other non-carbohydrate polymers, e.g., polyurethane layers or glutaraldehyde/albumin mixtures.

The term “hydrogel”, as used herein and in the context of the present invention, is a term being well known to a person skilled in the art and includes any network of covalently crosslinked polymer chains that are hydrophilic. It usually builds up a three-dimensional solid, consisting of hydrophilic polymer chains, being held together by specific crosslinkers. Because of the inherent crosslinkers, the structural integrity of the hydrogel network does not dissolve in water. Hydrogels are highly absorbent (they can contain over 90% water) natural or synthetic polymeric networks.

In some aspects of the invention, it may be useful to use different layers of polymers. These polymers may vary in composition, polarity, pore size and degree of crosslinking. These layers can be installed sequentially using processes described below.

In one embodiment of the invention, one or more enzymes can be added to the mixture of polymers. Furthermore, the viscosity of the resulting mixture can be adjusted by adding additional solvents, water, surfactants or other ingredients known to the one skilled in the art.

A thermally-induced, drop-spin gelation process allows application of geometrically precise layers onto sensor chip surfaces. This provides a modular fabrication system since one can control the crosslinking degree, linker polarity, linker length, and, correspondingly, the pore size. After mixing and drop-spin coating the non-viscous solutions of the individual gel components, formation of a covalent, non-degradable network can be induced in the layer by heating the sensor to mild temperatures (e.g., 40° C. for between 1 to 15 hours), which is compatible with maintaining enzymatic activity.

As used herein, a “covalent network” consists of at least two polymeric components, which are bonded together by covalent chemical bonds, i.e. not exclusively relying on e.g. electrostatic or van der Waals interactions (albeit these may contribute to forming the network). The term “non-degradable” refers to forming bonds that cannot be cleaved by enzymatic reactions (e.g., by peptidases or esterases) or autohydrolysis.

The solution can be dropped in the center of an electrode pattern set and then spun to spread across the target area. Multiple drops spread out over the pattern set can also be used. Layer thickness may be varied by changing the spin speeds (e.g., 1000-6000 rpm) with higher speeds producing thinner films. This process is compatible with microfabrication tooling, and the resulting layer is compatible with microfabrication vacuum systems and organic solvents. Thin layers between 20 nm to 1000 nm are achievable, although thicker layers can be achieved if desired. In general, thin electrode coating layers provide superior signal efficiency because diffusion time of generated reaction products to reach the electrode is shorter.

As shown herein, the drop-spin process and the various microfabrication processes allow multiple layers in multiple configurations to be built. These drop-spin hydrogels show good adhesion to repeated layers and to biosensor electrodes.

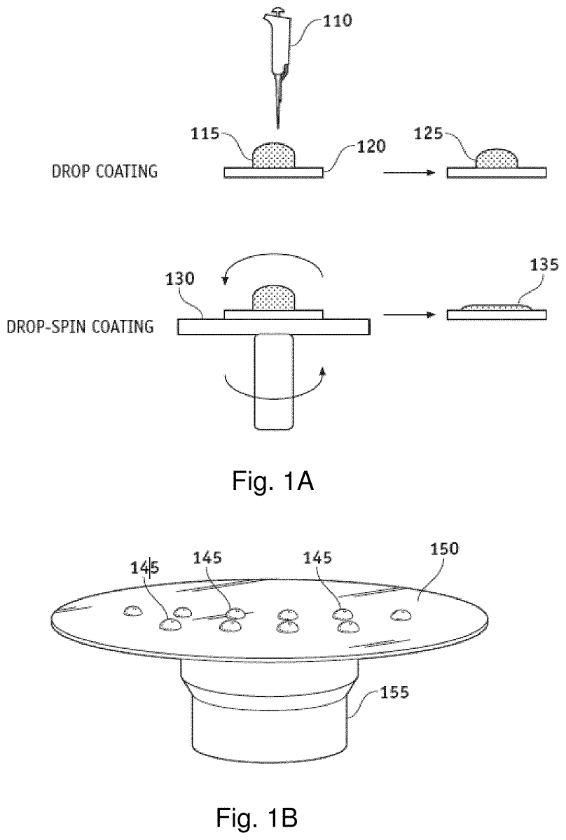

FIG. 1A shows an example of the drop-spin method. With the drop coating method, an applicator (110) (e.g., a pipette) applies a drop (115) of the coating (e.g., hydrogel) on the surface of the wafer (120), which over time spreads (125) to a thickness of 1-3 micrometers. For the drop-spin coating method, a spinning platform (130) causes the coating (135) to compress to a thickness of only 200-600 nm. An example drop size is 20 microliters (μL) from mix (e.g., pullulan plus enzyme, two component system). An example spin speed is 4000 rpm over 30 seconds. In some embodiments, the spin speed is in a range from 1000-6000 rpm. In some embodiments, the drop size is in the range of 2 μL to 100 μL. In some embodiments the drop size is over 100 μL. In some embodiments, the spinning starts at low speed (e.g., 500 rpm) for a first interval (e.g., for ten seconds) and then increases to a high speed (e.g., 4000 rpm) for a second interval (e.g., 30 seconds). For example, the lower speed can be less than 1000 rpm and the higher speed can be greater than 1000 rpm. After the drop-spin, the hydrogel can polymerize on top of the electrodes (e.g., in a 40-degree C oven).

FIG. 1B shows a wafer (150) with multiple drops (145) set on the rotating assembly (155) before the spin step.

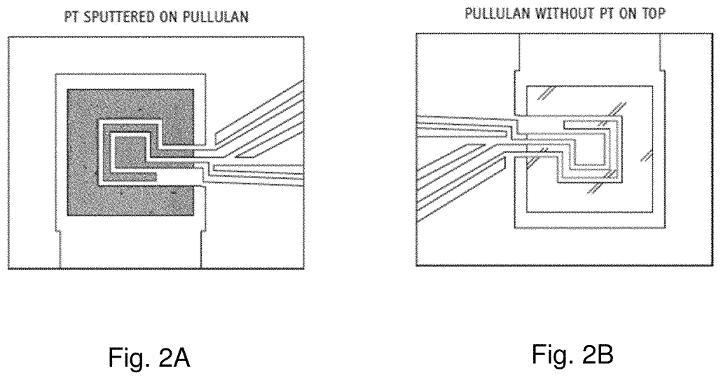

The drop-spin method is compatible with microelectronic methods for wafer processing. For examples, ultraviolet (UV) induced functionalization, vacuum, clearing/washing with organic solvents. FIGS. 2A and 2B show examples of platinum sputtered on pullulan deposited by the drop-spin method (FIG. 2A) and pullulan deposited by the drop-spin method without the platinum (FIG. 2B). The deposition process allows placing a thin layer of biopolymer exactly on the electrodes, along with a metal coating exactly on top of that. In some embodiments, the coating is in the range of 0.2 μL to 100 μL per sensor, or around 2-15 mL per 100 mm diameter wafer (preferably 3-10 mL). Also, small volumes can be used, i.e. from 0.1 μL to 10 μL. In some embodiments, the biopolymer layer is less than 600 nm thick. In some embodiments, the biopolymer layer is between 200-600 nm thick. In some embodiments, the biopolymer layer is between 200-400 nm thick. In some embodiments, the biopolymer layer is under 200 nm thick. In some embodiments, the layer over the biopolymer (e.g., platinum) is up to 50 nm thick. In some embodiments, the layer over the biopolymer is 50 nm to 100 nm thick. In some embodiments, the layer over the biopolymer is up to 100 nm thick. In some embodiments, the layer over the biopolymer is over 100 nm thick.

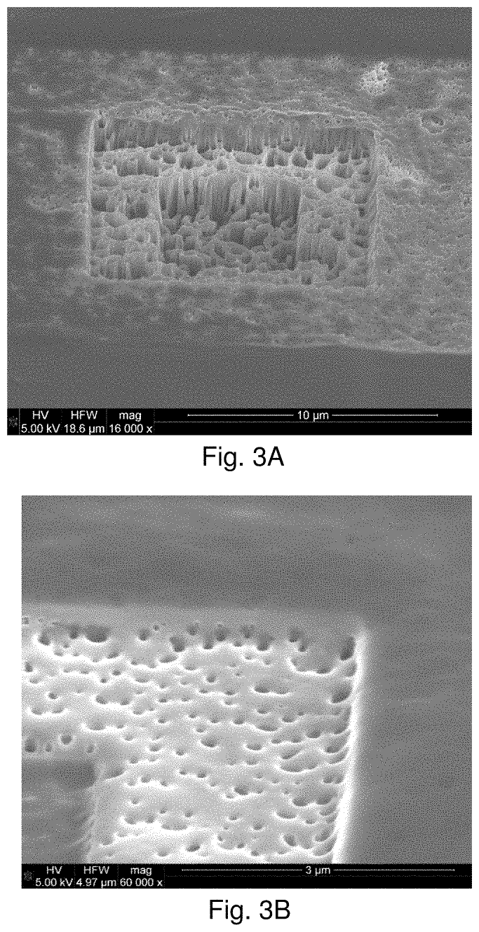

FIG. 3 shows an example scanning electron microscope image of a sensor with a pullulan-enzyme mix deposited by the drop-spin method. As shown, very thin layers are possible (e.g., 20 nm to 1 micrometer) with no deep 3D structure.

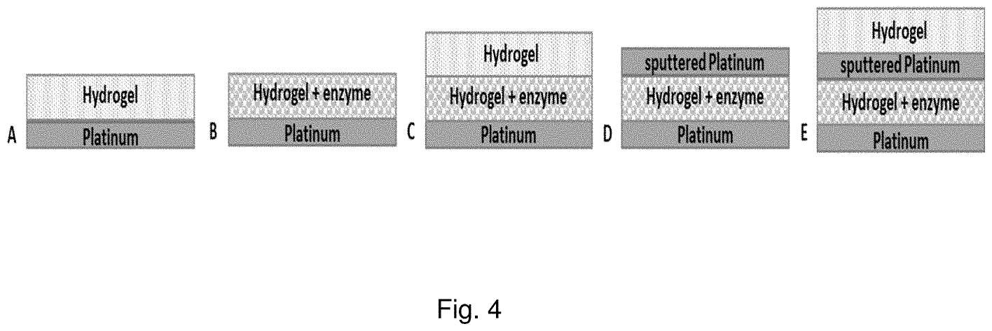

FIG. 4 shows examples of coating techniques. Panel A shows a single-layer hydrogel (e.g., pullulan) on an electrode (e.g., platinum on silicon/SiO₂). Panel B shows a single layer hydrogel with an enzyme immobilized in the hydrogel network. Panel C shows a two-layer coating, where the top layer can be a biopolymer layer without an enzyme, thereby acting as a diffusion or protection barrier. The layers can be fabricated by drop spin coating, particularly if a thin layer/multiple-layer is wanted—other fabrication methods can also be used as understood by one skilled in the art. Panel D shows a different two-layer system with sputtered platinum on the top layer. Panel E shows a three-layer system, where both a sputtered platinum layer and an enzyme-free hydrogel layer are on top of the hydrogel-enzyme layer. This gives an improved approach in terms of a body's immune response system, and so works well for an implantable chip. Even further, layers can be added depending on the desired properties and use.

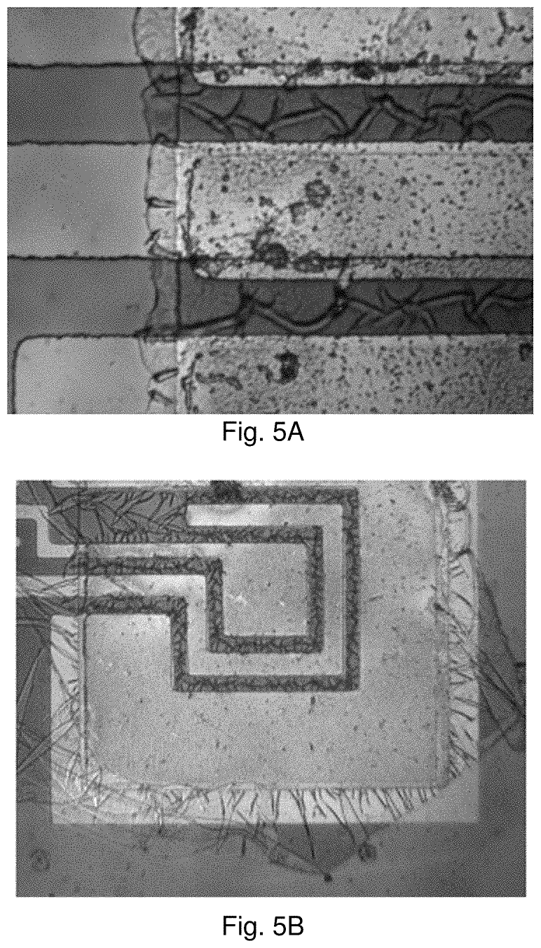

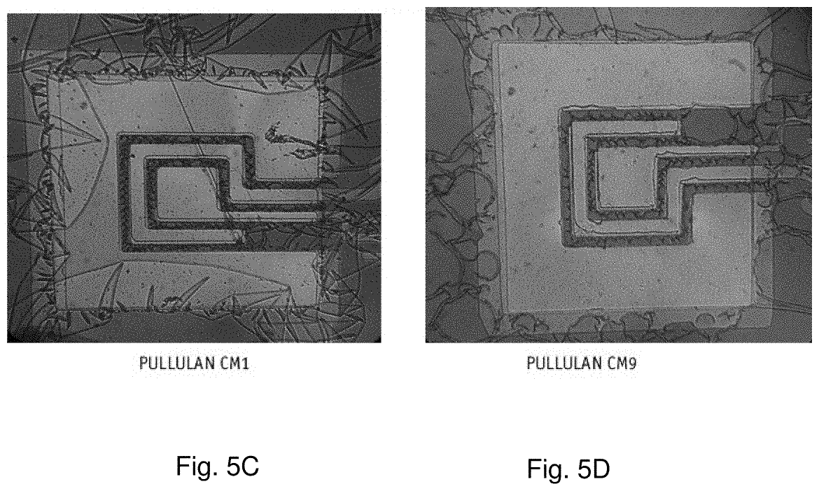

FIGS. 5A to 5D show examples of the adhesion between the hydrogel and electrode. In the example, the sensors (platinum electrode) were coated with pullulan-enzyme via drop-spin. Then they were incubated for two to three days in deionized water, in a chamber large enough to allow them to be completely covered by the deionized water. This washed away everything except the bound biopolymer and the immobilized enzyme. After removing the wafer from the chamber, all hydrogel areas not on top of the electrode were washed away. There is a defined area where the pullulan sticks very strongly to the electrode area. Photolithography and liftoff processing can be used to define where the electrodes are on the substrate. In other areas of the wafer (e.g., Si₃N₄), the pullulan is removed. The individual network properties and density depend on the nature of the used polymer and its degree of crosslinking. This can be illustrated by the optical appearance of electrodes covered with PCM1 and PCM9. Electrode adhesion properties, however, are comparable for the two differently crosslinked polymers. Depending on the specific biosensing question, differently modified materials can provide optimal coatings for the desired application, as understood by one skilled in the art. FIGS. 5C and 5D show examples of adhesion for once carboxymethylated (CM) 1 pullulan (FIG. 5C) and nine times carboxymethylated (CM9) pullulan (FIG. 5D).

In the context of the present invention, the carboxymethylation degree of pullulan is presented by the expression “PCM” followed by a number (e.g. PCM1, PCM3 and PCM5). This number characterizes the respective carboxymethylation degree, meaning the carboxymethyl-groups introduced into pullulan by applying the designated number of repetitive carboxymethylation reaction cycles. As used herein, CM1, CM2, CM3, etc. describe the respective carboxymethylation degree of a polysaccharide in general (without specifically referring to pullulan).

CM1 (or in case of pullulan PCM1) is considered a low modified biopolymer. CM9 (or PCM9) is considered a highly modified biopolymer. Higher modified biopolymers (PCM5-PCM9) have a short gelation time (2-5 h; 40 mg/ml material). Low modified biopolymers (PCM1-3) have a gelation time from 3-10 h (40 mg/ml material).

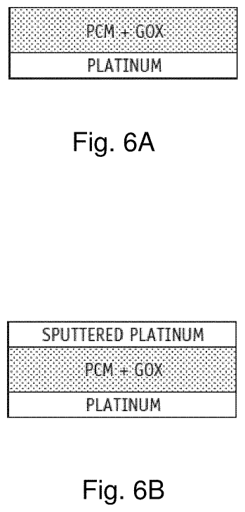

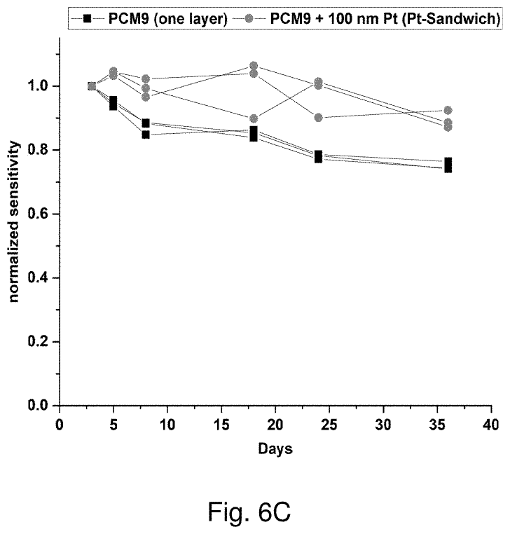

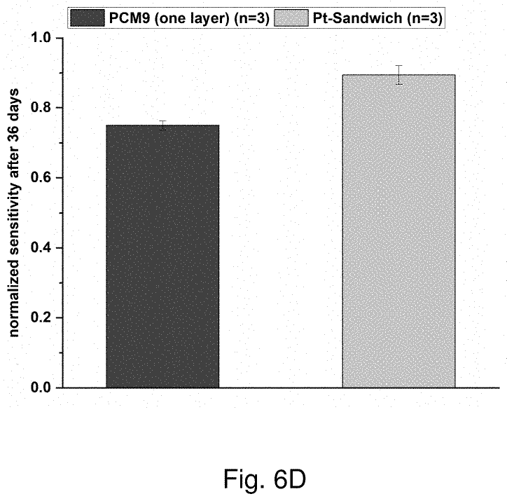

FIGS. 6A to 6D illustrate the longevity of the drop-spin deposited sensor system. In an example, a single wafer had two sensor types drop-spin deposited on it: a single layer pullulan CM9 (FIG. 6A) and a pullulan CM9 with top platinum layer (FIG. 6B). The hydrogel layers here were pullulan CM (PCM) with glucose oxidase (GOX) isolated in the PCM. The layers, deposited on electrode systems, were stored either in phosphate-buffered saline solution or in air. Note that with the drop-spin process, the enzyme and sensor are still active after dry storage at room temperature, which is not the case for previous methods. The glucose concentration ranged up to 42 mM. Both sensor types were exposed to a high vacuum, subsequent acetone incubation (1-2 hours) and were then washed with isopropyl alcohol prior to measurement. It can be seen that sensor functionality is maintained under these challenging conditions.

FIG. 6C shows the normalized sensitivity over time for the sensors. For FIGS. 6C, 6D, and 7-11, sensors 1, 2, 5, 6, and 7 are one-layer sensors (FIG. 6A, PCM9+GOX) and sensors 3, 4, 8, 9, and 10 are two-layer sensors (FIG. 6B, PCM9+GOX+100 nm Pt). The tests were performed on two wafers—one stored in PBS and one stored dry.

FIG. 6D shows the average sensitivity over 36 days for each. The one-layer (FIG. 6A) has an average sensitivity (normalized) of 0.75, while the two-layer (FIG. 6B) has a sensitivity of 0.89, due to the diffusion barrier effect.

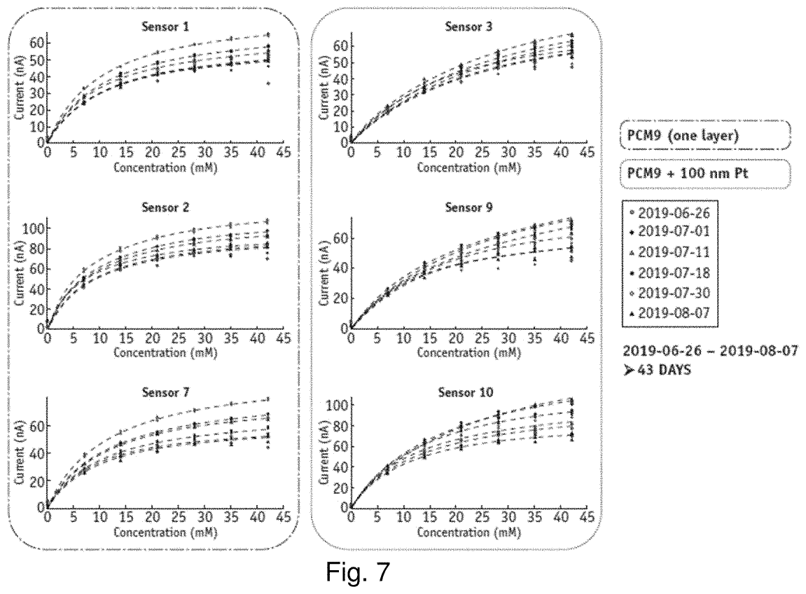

FIG. 7 shows concentration vs. current graphs for the sensors of the previous example (FIG. 6A and FIG. 6B) over 43 days. The wafer was stored in phosphate-buffered saline (PBS). The one-layer sensors (1, 2, and 7 here) have a different slope than the two-layer sensors (3, 9, and 10 here) and the initial gradient changed with time.

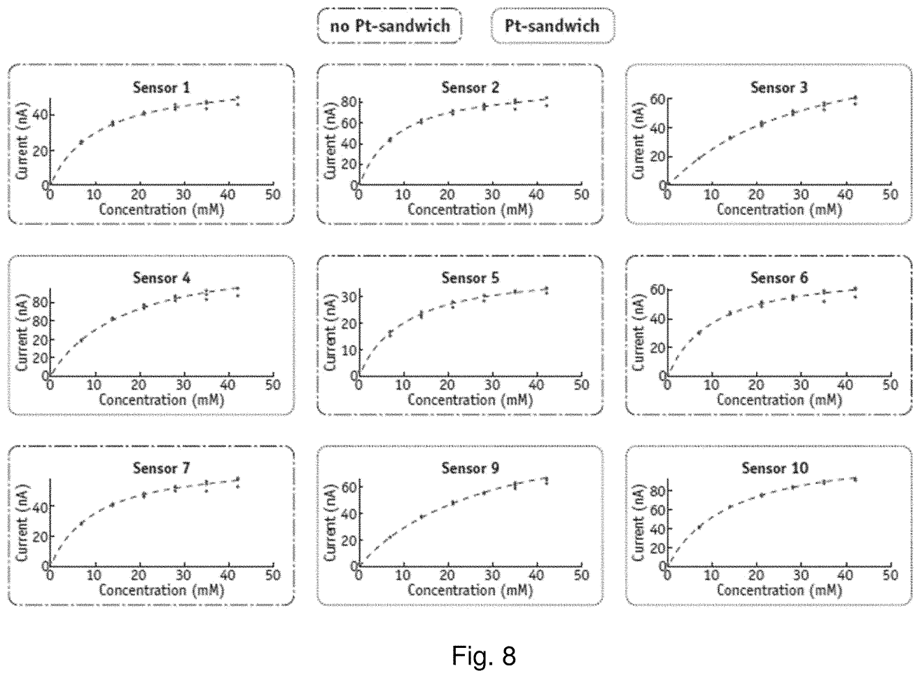

FIG. 8 also shows the difference between the sensor types (FIGS. 6A and 6B) in concentration vs. current. The two-layer sensors have a better (slower) initial gradient and a larger linear range.

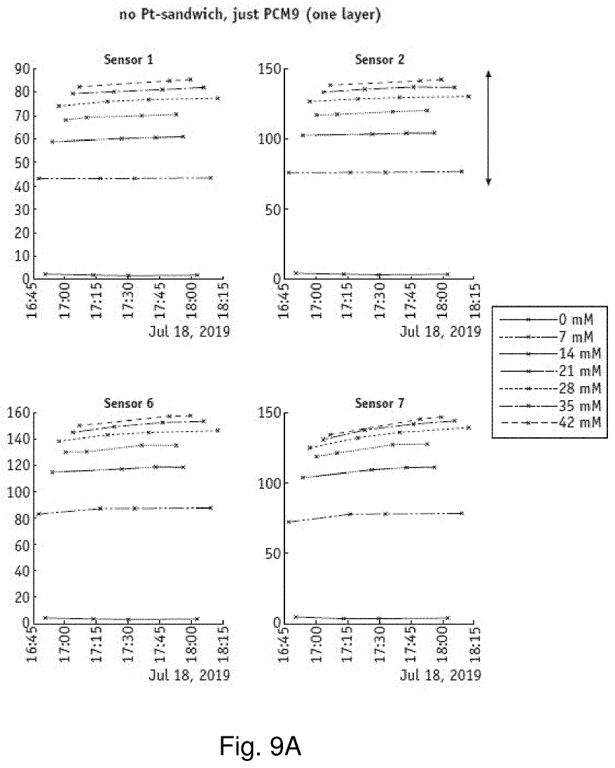

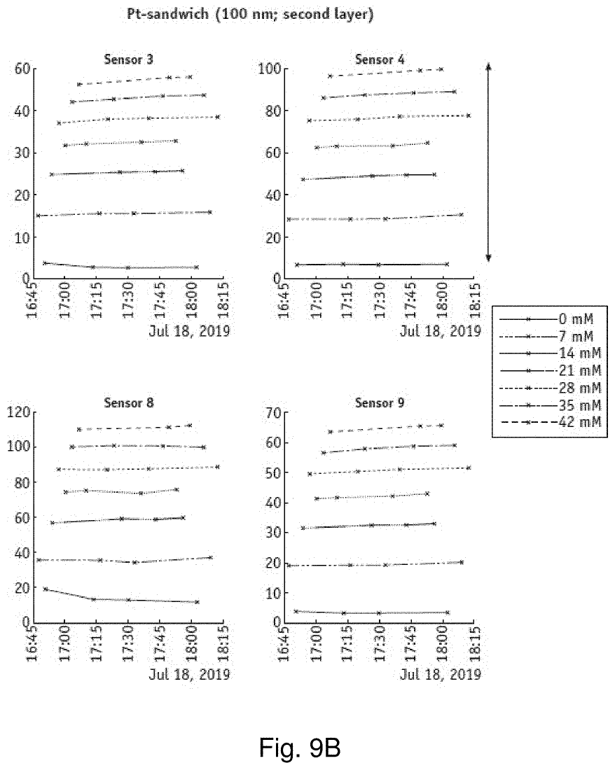

FIGS. 9A and 9B show an example comparison between the one-layer sensor (FIGS. 6A and 9A) and the two-layer sensor (FIGS. 6B and 9B), the two-layer with 100 nm thickness (platinum top layer) and both stored in PBS. As shown, the two-layer sensors show a better linear range. The observed current density is for example 10.5 A/m², which is obtained by dividing the measured current (longitudinal axis in nA) by the area of the working electrode, e.g. for measured 150 nA=10.5 A/m²).

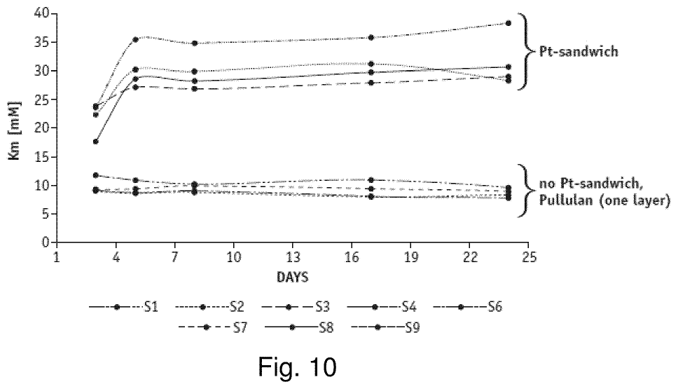

FIG. 10 shows a comparison of the one-layer (FIG. 6A) and two-layer “sandwich” (FIG. 6B) sensors Km (enzyme's substrate affinity) values from 3-24 days. The Km values are noticeably higher for the two-layer sensor than the one-layer sensors. The one-layer sensors had a Km value between 9-12 mM, while the two-layer sensors had Km values from 25-38 mM.

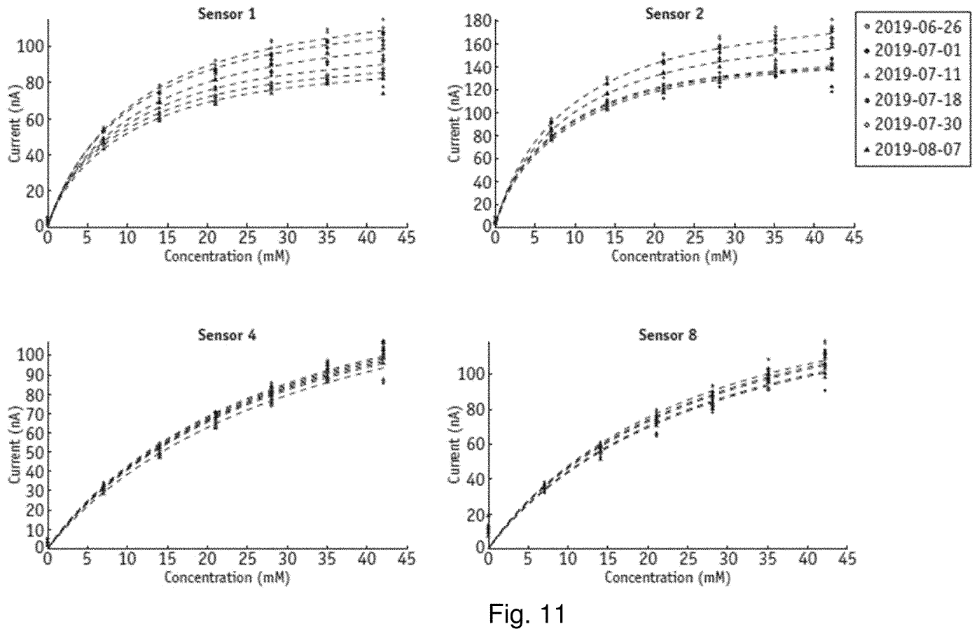

FIG. 11 shows example enzyme saturation curves (from sensors in the previous example). The data was taken over 24 days from a dry wafer. The two-layer sensors (4 and 8 here; see e.g., FIG. 6B) were 100 nm Pt diffusion barrier, 200-600 nm GOX hydrogel layer, 200 nm Pt conductive layer, 2000 nm SiO₂, and 0.5 mm Si substrate. The two-layer sensors show a larger linear range and so are more stable over time compared to one-layer sensors. The one-layer sensors (1 and 2 here; see e.g., FIG. 6A) are PCM9.

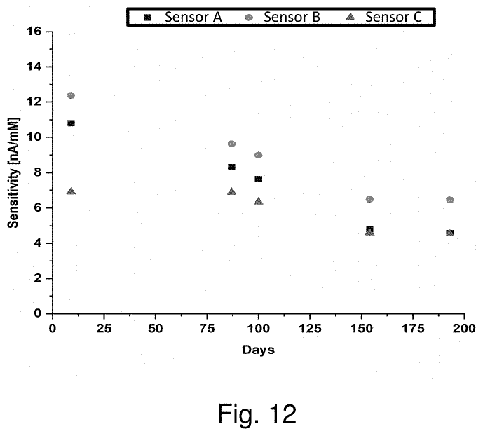

FIG. 12 shows an example graph of the long-term (e.g., 200 days) longevity of sensors being two-layer sensors having a 50 nm sputtered platinum top layer on a PCMS hydrogel (see e.g., FIG. 4C). The sensors were stored in 1× PBS at room temperature (rt).

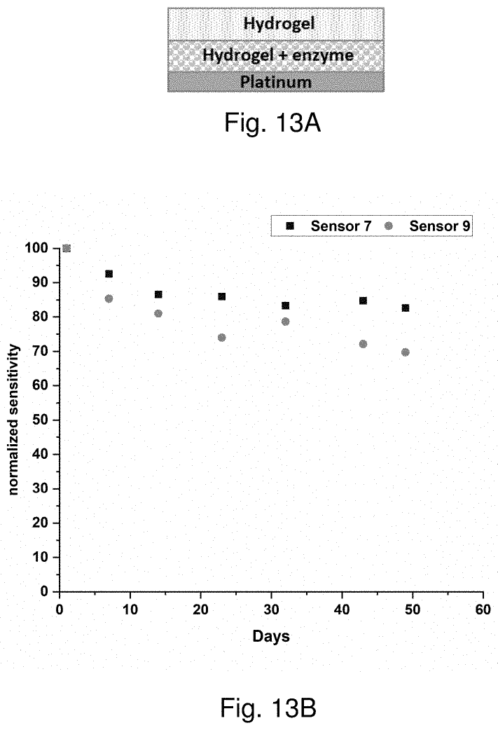

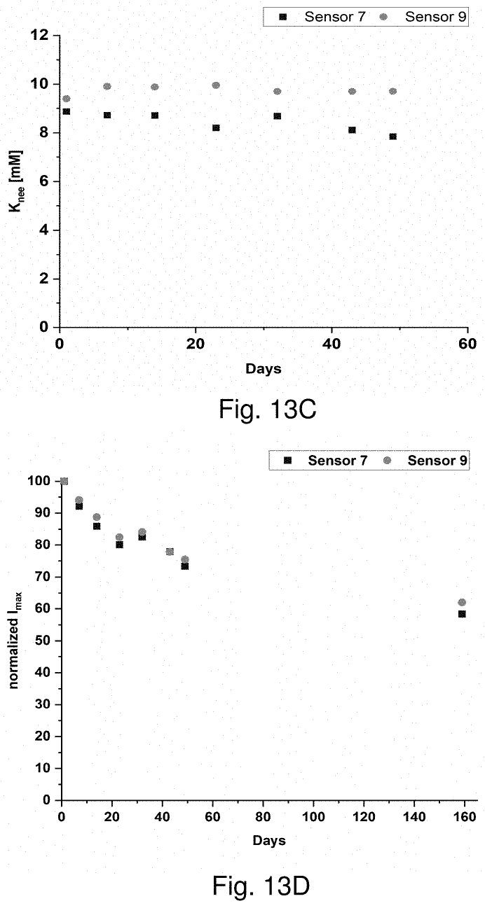

FIGS. 13A-13D show example data for two two-layer sensors (see sensors 7 and 9 of FIG. 4C). FIG. 13A shows the structure of the sensors—it is a two-layer sensor with a covering hydrogel (without enzyme) instead of a Pt sandwich. FIG. 13B shows the normalized sensitivity over time, showing a good stability of sensitivity over time. FIG. 13C shows Knee (K) values (mM) over time, also shown to be highly stable. FIG. 13D shows normalized maximum current over time, showing only a drop from 100 to 60 over 160 days. These results are from tests performed in a PBS buffer (pH 7.4) at room temperature. The hydrogel is PCMS (mid-range) and glucose oxidase is the enzyme.

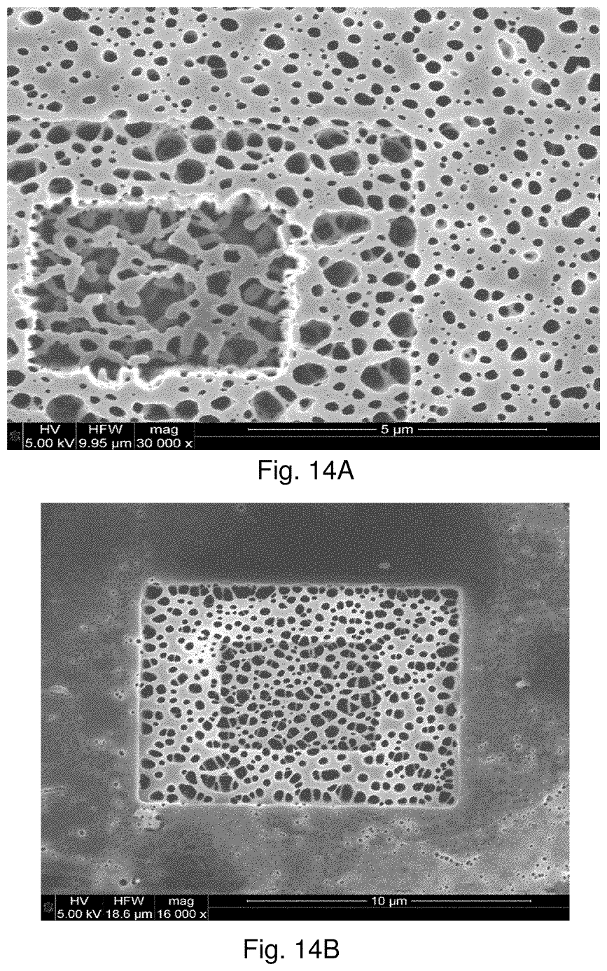

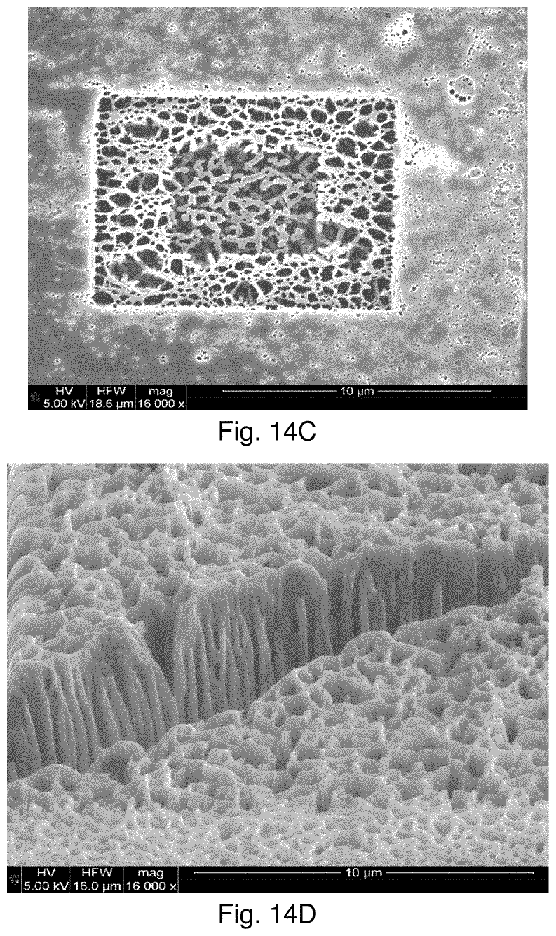

FIGS. 14A-14D show example scanning electron microscope (SEM) images of drop-coat (previous technique) applications. As shown, the drop-spin application method provides a thinner layer.

FIG. 14A shows a PCMS drop-coat with hydrogel, in distilled water (dH₂O). FIGS. 14B and 14C show a PCM9 drop-coat with hydrogel, in dH₂O. FIG. 14D shows a closeup of PCM6 drop-coat with hydrogel, in PBS.

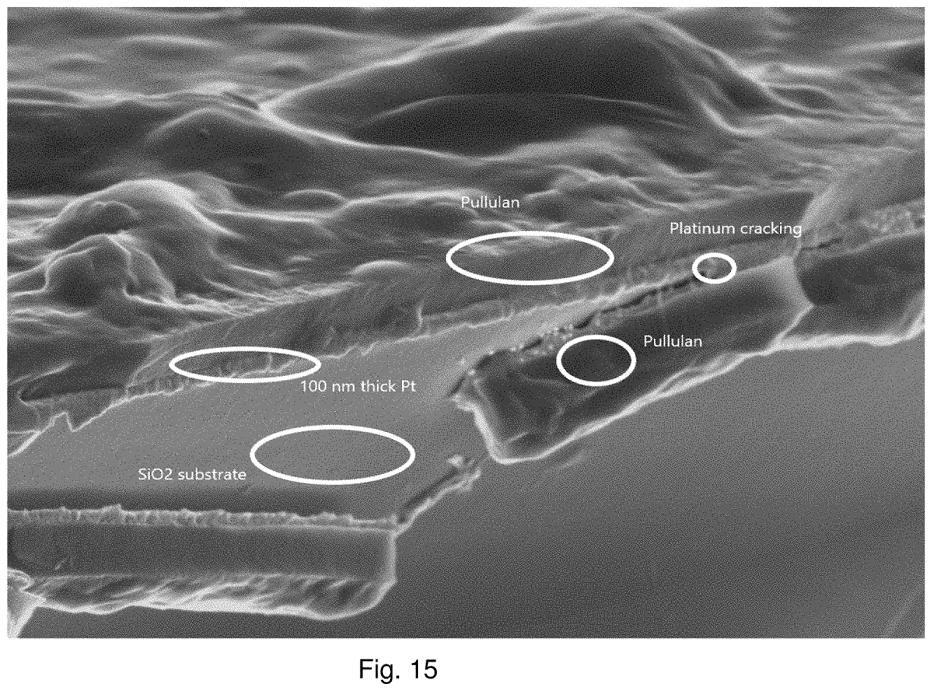

FIG. 15, in contrast to FIGS. 14A-14D, shows an example of a SEM image of the different layers and estimated layer thicknesses for the drop-spin application with hydrogel (Pullulan), in dH₂O.

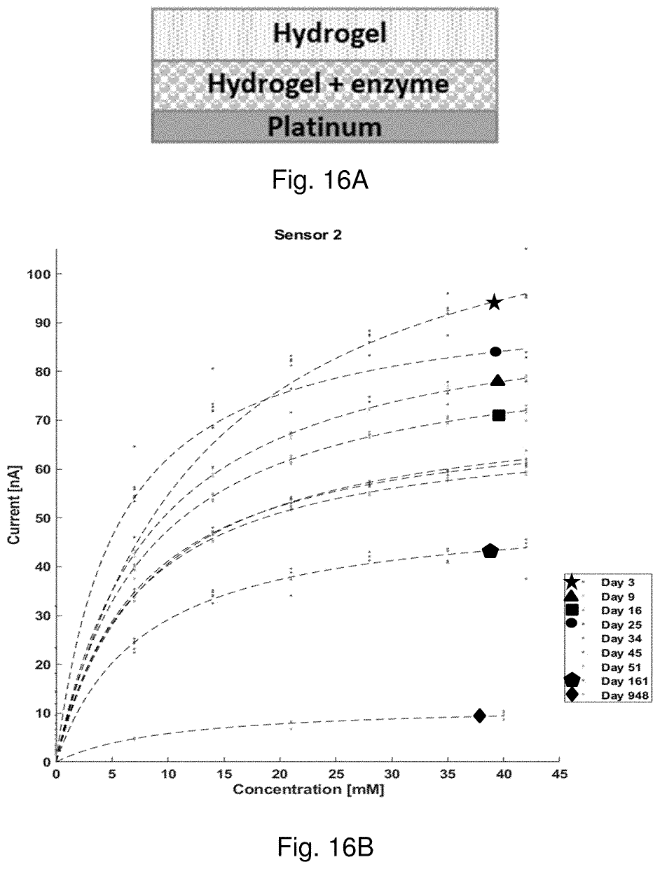

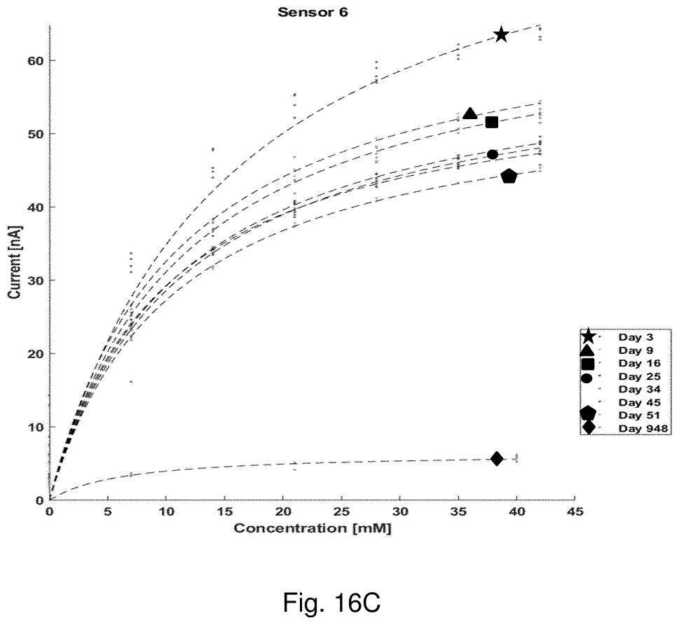

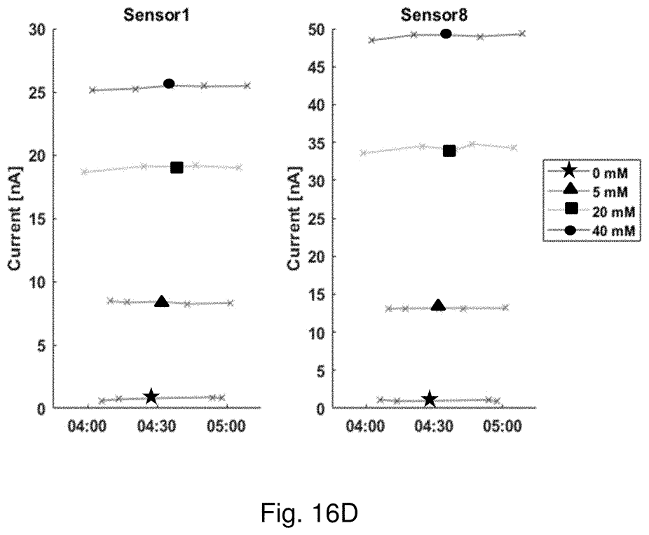

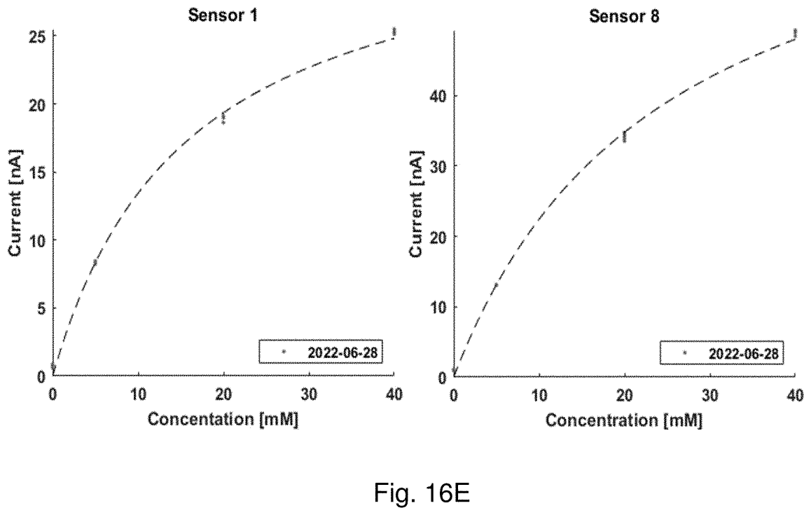

FIGS. 16A-16D show exemplary data for two-layer sensors (see e.g., FIG. 4C). FIG. 16A shows the composition of the sensors—the data is referring to a two-layer sensor with a covering hydrogel layer (without enzyme). FIGS. 16B and 16C shows enzyme saturation curves over a period of 948 days. Graphs 16D and 16EE show the sensitivity of the sensors on day 1148 and, therefore, illustrate the robustness of the sensors and the stabilizing effect of the hydrogel on the enzyme. Both hydrogel layers consist of PCMS, glucose oxidase was used as the enzyme. Sensors were stored in PBS (pH 7.4) at room temperature over the measurement period.

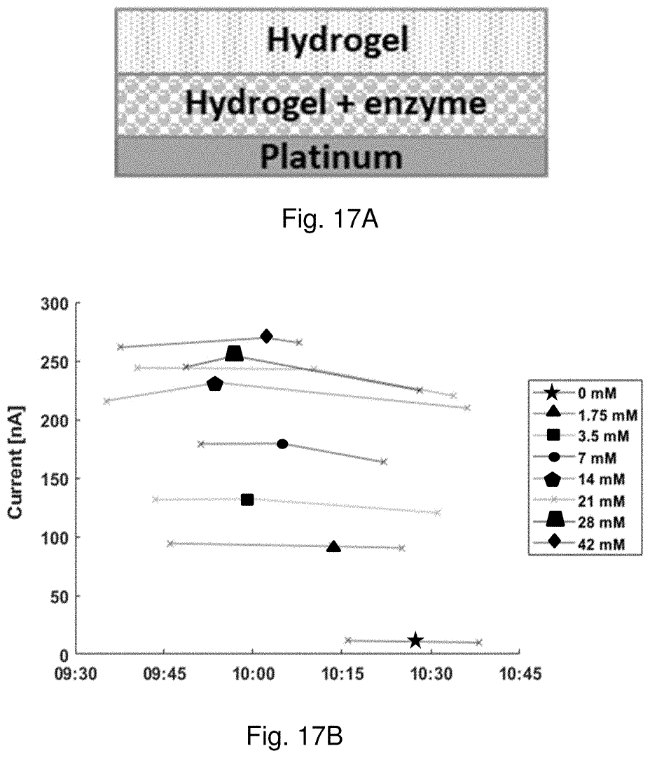

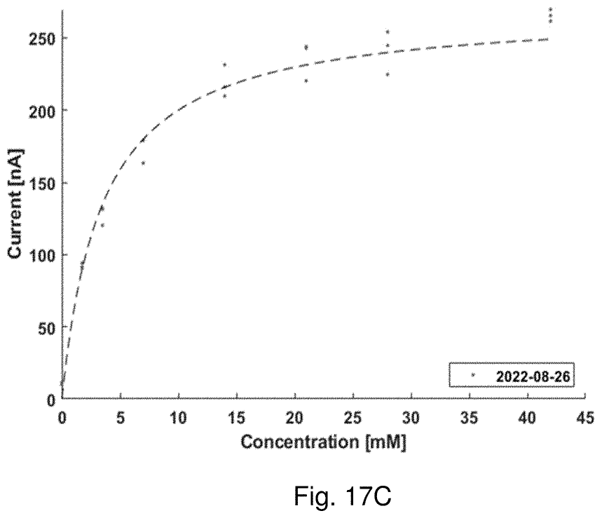

FIGS. 17A-17C show exemplary data for two-layer sensors (see e.g., FIG. 4C). FIG. 17A shows the composition of the sensors—the data is referring to a two-layer sensor with a covering hydrogel layer (without enzyme). Graphs 17B-17C show the sensitivity of the sensor over a high glucose concentration range (0-42 mM). A particularly high sensitivity of 49 nA/mM was determined for the sensor. The first hydrogel layer is composed of a PCM4 hydrogel with immobilized glucose oxidase and the second hydrogel layer consists of an alginate hydrogel. Sensor was stored in PBS (pH 7.4) at 37° C. for one day.

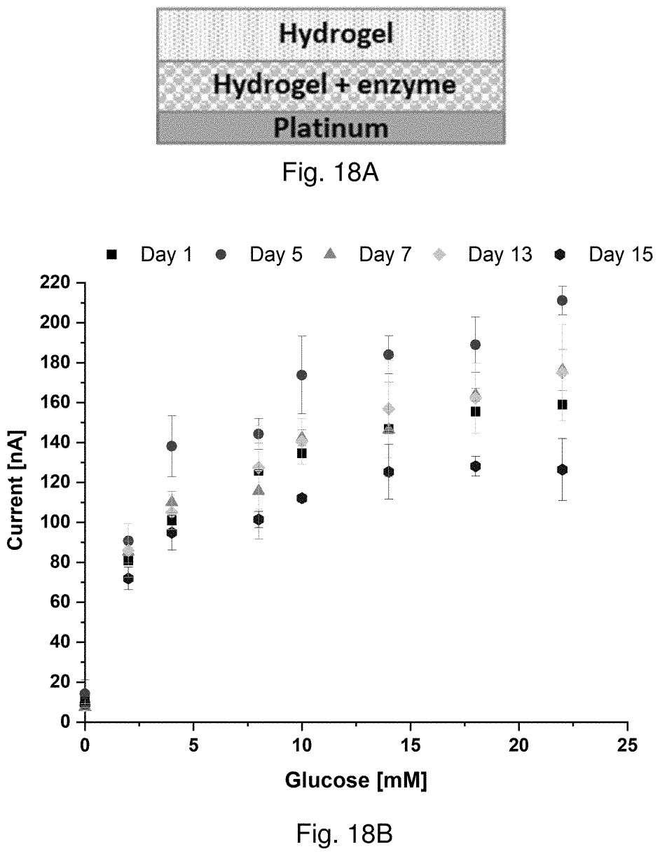

FIGS. 18A-18B show example data for two-layer sensors (see e.g., FIG. 4C). The composition of the sensor is depicted in FIG. 18A—the data is referring to a two-layer sensor with a covering hydrogel (without enzyme). Graph 18B shows the enzyme saturation curve over 15 days. Over a period of 15 days of continuous incubation at 37° C., these results demonstrate the stability and longevity of the immobilized glucose oxidase and the positive benefit of the second hydrogel layer of alginate. The first hydrogel layer is composed of a PCM4 hydrogel with immobilized glucose oxidase and the second hydrogel layer consists of an alginate hydrogel. Sensor was stored in PBS (pH 7.4) at 37° C. for 15 days.

Example Hydrogel

The present drop-spin method can be used with a method to immobilize enzymes in a biocompatible hydrogel that does not require a covalent binding of one or more enzyme(s) (identified in this Example as “a biocompatible hydrogel”). This hydrogel provides a perfect stabilizing environment for the enzyme, resulting in a better lifetime and enzyme stability. This hydrogel protects the enzyme from biofouling and body immune response. This hydrogel is described in EPO application No. EP21202802.1 titled HYDROGEL FOR IMMOBILIZATION OF ONE OR MORE ENZYME(S) AND METHOD FOR PREPARING THE SAME invented by Oliver Plettenburg et al. and filed on Oct. 15, 2021, and PCT/EP2022/078635 with the same title and filed on Oct. 14, 2022 and published as WO/______, all of which are incorporated by reference herein.

A method for preparing such a biocompatible hydrogel can comprise the following steps:
a) Providing a first polysaccharide and a second polysaccharide, preferably wherein the first polysaccharide and/or the second polysaccharide being independently from each other selected from the group consisting of pullulan, alginate, cellulose, hyaluronan, dextran, lichenin, lentinan, and mixtures thereof, with n being the number of the monomeric repeating unit of the first and/or second polysaccharide and with n being an integer from 10 to 10000,
b) optionally carboxymethylation of at least one OH-group of the first and/or second polysaccharide;
c) functionalization of the first polysaccharide with one or more linker unit(s) of the structure -A-X, when the monomeric repeating unit of the first polysaccharide not comprises a carboxylic acid residue, or functionalization of the first polysaccharide with one or more linker unit(s) of the structure -X, when the monomeric repeating unit of the first polysaccharide comprises a carboxylic acid residue, wherein A is —(CH₂)_{d}—C(O)— or —C(O)—NH—, wherein d is an integer from 1 to 3, wherein X is selected from the group consisting of —NH—(CH₂)ᵣ—N₃, —NH—(CH₂CH₂O)ₛ—CH₂CH₂N₃, and —NH—(CH₂—CH₂—C(O))ₜ—CH₂—CH₂—N₃, with r being an integer from 2 to 20, s being an integer from 1 to 15, and t being an integer from 1 to 15; and
   functionalization of the second polysaccharide with one or more linker unit(s) of the structure -A′-Y, when the monomeric repeating unit of the second polysaccharide not comprises a carboxylic acid residue, or functionalization of the second polysaccharide with one or more linker unit(s) of the structure -Y, when the monomeric repeating unit of the second polysaccharide comprises a carboxylic acid residue, wherein A′ is —(CH₂)_{d}—C(O)— or —C(O)—NH—, wherein d is an integer from 1 to 3,

wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,

wherein Q is

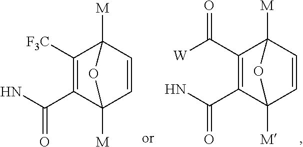

wherein M, M′=H or Me, and

wherein W=OMe, OEt, OH, NH₂ or NHMe,
d) optionally addition of one or more enzyme(s) to the mixture formed by steps a)-c),
e) incubation of the mixture formed by steps a)-d) in an aqueous medium at a temperature being in the range from 25° C. to 70° C., preferably 40° C., for at least 1 hour, preferably for 1 to 10 hours.

The biocompatible hydrogel can comprise:
a crosslinked polymer comprising the following structure:
a first polysaccharide and a second polysaccharide,
preferably wherein the first polysaccharide and/or the second polysaccharide being independently selected from the group consisting of pullulan, alginate, cellulose, hyaluronan, dextran, lichenin, lentinan, and mixtures thereof, with n being the number of the monomeric repeating unit of the first and/or second polysaccharide, with n being an integer from 10 to 10000,
one or more linker unit(s), which link the first polysaccharide with the second polysaccharide, wherein the structure of the one or more linker unit(s) is
-A-Z₁-B-Z₂-A′-,
wherein A and A′ are independently from each other —(CH₂)_{d}—C(O)— or —C(O)—NH—, wherein d is an integer from 1 to 3,
wherein Z₁ is selected from the group consisting of —O—, —(CH₂)ᵣ—, —NH—(CH₂CH₂O)ₛ—CH₂CH₂—, and —NH—(CH₂—CH₂—C(O))ₜ—CH₂—CH₂—, with d being an integer from 1 to 4, r being an integer from 2 to 20, s being an integer from 1 to 15, and t being an integer from 1 to 15; and,

wherein Z₂ is selected from the group consisting of —O—, —(CH₂)ᵣ—, —NH—(CH₂CH₂O)ₛ—CH₂CH₂— and —NH—(CH₂—CH₂—C(O))ₜ—CH₂—CH₂—,

wherein B is

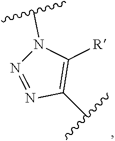

and

wherein R′ is selected from the group consisting of —CF₃, —C(O)—OMe, —C(O)—OEt, —C(O)—OH, —C(O)—NH₂ and —C(O)—NHMe, for non-covalent immobilization of one or more enzyme(s).

As used herein, the term “biocompatible” means, especially in connection with a hydrogel, that the respective material being called or assessed as being biocompatible has the quality of not having toxic or injurious effects on biological systems, that it has the ability to perform its desired function without eliciting any undesirable local or systemic effects in the recipient, but generating the most appropriate beneficial response in that specific situation, or the ability to exist in harmony with tissue without causing deleterious changes. Preferable properties of biocompatible materials are reduced inflammation and immunological response and/or low/limited fibrotic encapsulation.

The first and/or second polysaccharide may be optionally carboxymethylated in step b), wherein at least one OH-group of the first and/or second polysaccharide as defined herein above may be carboxymethylated. In one embodiment, the carboxymethylation is preferably carried out, when the first and/or second polysaccharide is/are pullulan or dextran.

As used herein, the term “functionalization” or “functionalized” means in general the addition of specific functional groups to afford the compound new, desirable properties, e.g. the addition of a linker unit or linker units to the existent polysaccharide structure.

Functionalization of the second polysaccharide as defined above in step c) may be with one or more linker unit(s) of the structure -A′-Y, when the monomeric repeating unit of the second polysaccharide not comprises a carboxylic acid residue, or functionalization of the second polysaccharide as defined above in step c) may be with one or more linker unit(s) of the structure -Y, when the monomeric repeating unit of the second polysaccharide comprises a carboxylic acid residue, wherein A′ is —(CH₂)_{d}—C(O)— or —C(O)—NH—, wherein d is an integer from 1 to 3, wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,

wherein Q is

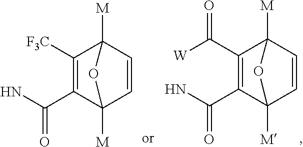

wherein M, M′=H or Me, and

wherein W=OMe, OEt, OH, NH₂ or NHMe.

As used in the present invention, Q may be

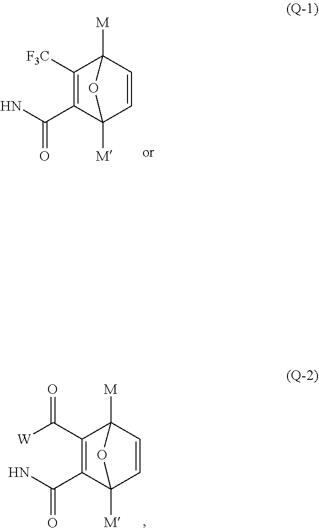

wherein M, M′=H or Me, and

wherein W=OMe, OEt, OH, NH₂ or NHMe. If, for the structural formula

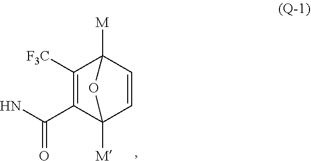

the structural formula

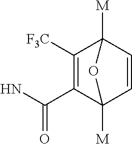

is used elsewherein herein, this does not mean for the latter mentioned structural formula that both M have to be the same (each H or each Me). Rather, the present invention also comprises that, in one embodiment both M can be H, in one embodiment both M can be Me, and in one embodiment one M can be H and the other M can be Me (independent from the position of M, two possibilities for one M being H and the other M being Me). Thus, in the context of the present invention, the two structural formulas

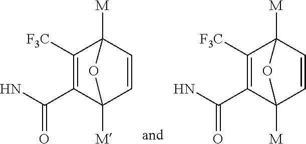

can be used interchangeably herein.

Moreover, in the context of the present invention, it is absolutely clear for the person skilled in the art, with regard to the functionalization of the second polysaccharide with one or more linker unit(s) of the structure -A′-Y,

wherein Y is selected from the group consisting of —NH—(CH₂)ᵣ-Q, —NH—(CH₂CH₂O)ₛ—CH₂CH₂Q and —NH—(CH₂—CH₂—C(O))ₜ—CH₂—CH₂-Q,

wherein Q is

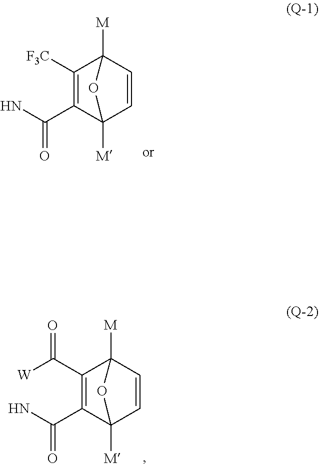

wherein M, M′=H or Me, and

wherein W=OMe, OEt, OH, NH₂ or NHMe, that the linking of Q within —NH—(CH₂)ᵣ-Q, —NH—(CH₂CH₂O)ₛ—CH₂CH₂Q or —NH—(CH₂—CH₂—C(O))ₜ—CH₂—CH₂-Q, is via the dashed line given in the structural formulas

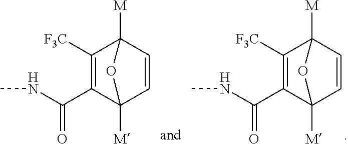

In one embodiment, in step d), optionally the addition of one or more enzyme(s) to the mixture formed by the steps a)-c) may be carried out. In this embodiment, any enzyme is in principle possible.

In step e), the incubation of the mixture formed by steps a)-d) is carried out in an aqueous medium at a temperature being in the range from 25° C. to 70° C. for at least one hour. It is more preferred that in step e), the incubation of the mixture formed by steps a)-d) is carried out in an aqueous medium at a temperature being in the range from 25° C. to 70° C. for 1-15 hours. It is even more preferred that in step e), the incubation of the mixture formed by steps a)-d) is carried out in an aqueous medium at a temperature being in the range from 25° C. to 70° C. for 1-10 hours.

It is preferred that in step e), the incubation of the mixture formed by steps a)-d) is carried out in an aqueous medium at a temperature being in the range from 30° C. to 60° C. for at least one hour. It is more preferred that in step e), the incubation of the mixture formed by steps a)-d) is carried out in an aqueous medium at a temperature being in the range from 30° C. to 60° C. for 1-15 hours. It is even more preferred that in step e), the incubation of the mixture formed by steps a)-d) is carried out in an aqueous medium at a temperature being in the range from 30° C. to 60° C. for 1-10 hours.

It is further preferred that in step e), the incubation of the mixture formed by steps a)-d) is carried out in an aqueous medium at a temperature being in the range from 35° C. to 50° C. for at least one hour. It is more preferred that in step e), the incubation of the mixture formed by steps a)-d) is carried out in an aqueous medium at a temperature being in the range from 35° C. to 50° C. for 1-15 hours. It is even more preferred that in step e), the incubation of the mixture formed by steps a)-d) is carried out in an aqueous medium at a temperature being in the range from 35° C. to 50° C. for 1-10 hours.

It is further preferred that in step e), the incubation of the mixture formed by steps a)-d) is carried out in an aqueous medium at a temperature being in the range from 35° C. to 45° C. for at least one hour. It is more preferred that in step e), the incubation of the mixture formed by steps a)-d) is carried out in an aqueous medium at a temperature being in the range from 35° C. to 45° C. for 1-15 hours. It is even more preferred that in step e), the incubation of the mixture formed by steps a)-d) is carried out in an aqueous medium at a temperature being in the range from 35° C. to 45° C. for 1-10 hours.

It is more preferred that in step e), the incubation of the mixture formed by steps a)-d) is carried out in an aqueous medium at a temperature of about 40° C. for at least one hour. It is even more preferred that in step e), the incubation of the mixture formed by steps a)-d) is carried out in an aqueous medium at a temperature of about 40° C. for 1-15 hours. It is even more preferred that in step e), the incubation of the mixture formed by steps a)-d) is carried out in an aqueous medium at a temperature of about 40° C. for 1-10 hours.

It is further preferred that in step e), the incubation of the mixture formed by steps a)-d) is carried out in an aqueous medium at a temperature being in the range from 30° C. to 60° C. for 4 to 10 hours. It is further preferred that in step e), the incubation of the mixture formed by steps a)-d) is carried out in an aqueous medium at a temperature being in the range from 35° C. to 50° C. for 4 to 10 hours. It is further preferred that in step e), the incubation of the mixture formed by steps a)-d) is carried out in an aqueous medium at a temperature being in the range from 35° C. to 45° C. for 4 to 10 hours. It is more preferred that in step e), the incubation of the mixture formed by steps a)-d) is carried out in an aqueous medium at a temperature of about 40° C. for 4 to 10 hours.

It is further preferred that in step e), the incubation of the mixture formed by steps a)-d) is carried out in an aqueous medium at a temperature being in the range from 30° C. to 60° C. for 4 to 6 hours. It is further preferred that in step e), the incubation of the mixture formed by steps a)-d) is carried out in an aqueous medium at a temperature being in the range from 35° C. to 50° C. for 4 to 6 hours. It is further preferred that in step e), the incubation of the mixture formed by steps a)-d) is carried out in an aqueous medium at a temperature being in the range from 35° C. to 45° C. for 4 to 6 hours. It is more preferred that in step e), the incubation of the mixture formed by steps a)-d) is carried out in an aqueous medium at a temperature of about 40° C. for 4 to 6 hours.

It is further preferred that in step e), the incubation of the mixture formed by steps a)-d) is carried out in an aqueous medium at a temperature being in the range from 30° C. to 60° C. for 6 to 8 hours. It is further preferred that in step e), the incubation of the mixture formed by steps a)-d) is carried out in an aqueous medium at a temperature being in the range from 35° C. to 50° C. for 6 to 8 hours. It is further preferred that in step e), the incubation of the mixture formed by steps a)-d) is carried out in an aqueous medium at a temperature being in the range from 35° C. to 45° C. for 6 to 8 hours. It is more preferred that in step e), the incubation of the mixture formed by steps a)-d) is carried out in an aqueous medium at a temperature of about 40° C. for 6 to 8 hours.

This method of preparing a biocompatible hydrogel may comprise with step e) a step comprising thermo-induced gelation, which e.g. allows formation of specific form factors e.g. by complete bubble-free filling of a suitably formed mould or by dropping a mixture of the two non-viscous solutions of the individual components into a lipophilic organic medium to form droplets of defined diameter. Formation of a covalent, non-degradable network of a biocompatible hydrogel can be induced by heating to mild temperatures as described above, which is compatible with maintaining enzymatic activity, when an enzyme is encapsulated therein. The method may comprise crosslinking via 1,3-dipolar cycloaddition, thermo-gelation under very mild reaction conditions (e.g., 40° C. in aqueous media) with no side reaction and no toxic reagent (e.g. glutaraldehyde). Optimization of pore sizes of the biocompatible hydrogel is possible by feasible adjustment of parameters and adjusting the degree of optional carboxymethylation. If an enzyme is added as described in step d), the one or more enzyme(s) will be immobilized in the produced biocompatible hydrogel, wherein the one or more enzyme(s) is/are then significantly longer stable and active than the free enzyme in solution at ambient or elevated temperature, e.g. body temperature, 37° C. Unstable sensitive enzymes, like alcohol oxidase, or glucose oxidase, have better lifetime performances under these conditions. This embodiment is also applicable to other sensitive enzymes. Such a produced biocompatible hydrogel can be stored dry without losing higher amounts of enzyme activity. Further, no or little leaching of enzyme can be achieved. Such hydrogels prepared according to this method can be suspended in aqueous or organic solvents, while maintaining enzymatic activity (e.g., acetone). The viscosity of the individual components, as well as of the mixture can be easily adjusted.

In one embodiment of the method of preparing a biocompatible hydrogel, the first polysaccharide and the second polysaccharide are independently from each other selected from the group consisting of pullulan, alginate, cellulose, hyaluronan, dextran, lichenin, lentinan and mixtures thereof.

Pullulan is a polysaccharide polymer consisting of maltotriose units. Three glucose units of maltotriose are connected by an α-1,4-glycosidic bond, whereas consecutive maltotriose units are connected to each other by an α-1,6-glycosidic bond. Pullulan may be produced from starch by the fungus Aureobasidium pullulans. It may be mainly used by the cell to resist desiccation and predation. The presence of this polysaccharide also facilitates diffusion of molecules both into and out of the cell. In the context of the present invention, the respective monomeric repeating unit for pullulan has the structure of

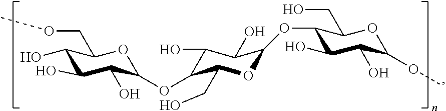

with n being the number of said monomeric repeating unit of pullulan, and with n being an integer from 10 to 10000.

Alginic acid, also called algin, is a polysaccharide distributed widely in the cell walls of brown algae that is hydrophilic and forms a viscous gum, when being hydrated. Alginic acid is a linear copolymer with homopolymeric blocks of (1-4)-linked β-D-mannuronate (M) and its C-5 epimer α-L-guluronate (G) residues, respectively, are covalently linked together in different sequences or blocks. The monomers may appear in homopolymeric blocks of consecutive G-residues (G-blocks), consecutive M-residues (M-blocks) or alternating M- and G-residues (MG-blocks). With metals, such as sodium and calcium, its salts are known as alginates. In the context of the present invention, the respective monomeric repeating unit for alginate has the structure of

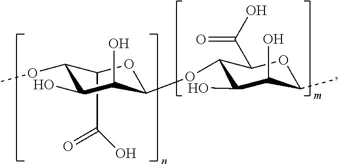

with n and m being the number of the monomeric repeating unit of alginate, and with n and m being each independently from each other an integer in the range from 10 to 10000.

Hyaluronic acid (abbreviated HA; conjugate base: hyaluronate), also called hyaluronan, is an anionic, non-sulfated glycosaminoglycan distributed widely throughout connective, epithelial and neural tissues. It is unique among glycosaminoglycans in that it is non-sulfated, forms in the plasma membrane instead of the Golgi apparatus and can be very large. Hyaluronic acid is a polymer of disaccharides, themselves composed of D-glucuronic acid and N-acetyl-D-glucosamine, linked via alternating β-(1→4) and β-(1→3) glycosidic bonds. In the context of the present invention, the respective monomeric repeating unit for hyaluronan has the structure of

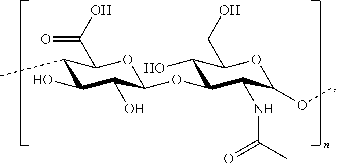

with n being the number of said monomeric repeating unit of hyaluronan, and with n being an integer from 10 to 10000.

Dextran is a complex branched glucan (polysaccharide derived from the condensation of glucose). IUPAC defines dextrans as “branched poly-α-D-glucosides of microbial origin having glycosidic bonds predominantly C-1→C-6”. Dextran chains are of varying lengths (from 3 to 2000 kilodaltons). The polymer main chain consists of α-1,6-glycosidic linkages between glucose monomers, with random branches from α-1,3-linkages. This characteristic branching distinguishes a dextran from a dextrin, which is a straight chain glucose polymer tethered by α-1,4 or α-1,6-linkages. In the context of the present invention, the respective monomeric repeating unit for dextran has the structure of

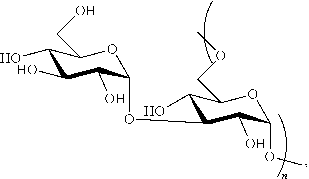

with n being the number of said monomeric repeating unit of dextran, and with n being an integer from 10 to 10000.

Lichenin, also known as lichenan or moss starch, is a complex glucan occurring in certain species of lichens. It is chemically a mixed-linkage glycan, consisting of repeating glucose units linked by β-1,3- and β-1,4-glycosidic bonds. In the context of the present invention, the respective monomeric repeating unit for lichenin has the structure of

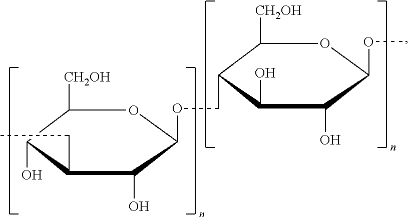

with n being the number of said monomeric repeating unit of lichenin, and with n being an integer from 10 to 10000.

Lentinan is a polysaccharide isolated from the fruit body of the shiitake mushroom. Chemically, lentinan is a β-1,3-beta-glucan with β-1,6-branching. In the context of the present invention, the respective monomeric repeating unit for lentinan has the structure of

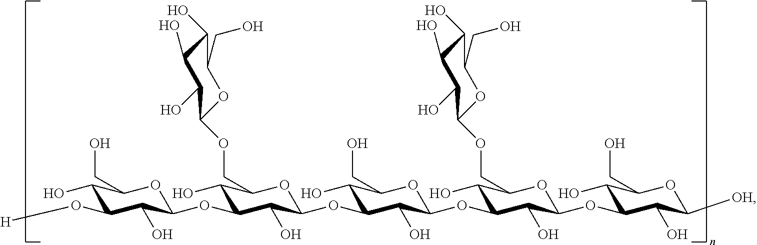

with n being the number of said monomeric repeating unit of lentinan, and with n being an integer from 10 to 10000.

In one embodiment of the method of preparing a biocompatible hydrogel, the first and/or second polysaccharide is dextran or pullulan and carboxymethylation of at least one OH-group of dextran or pullulan is carried out in step b). It is preferred for this embodiment that the carboxymethylation of at least one OH-group of dextran or pullulan is carried out in step b) at C₆ of dextran and/or pullulan. It is preferred for this embodiment that the carboxymethylation of at least one OH-group of dextran or pullulan is carried out in step b) at C₂ of dextran and/or pullulan. It is preferred for this embodiment that the carboxymethylation of at least one OH-group of dextran or pullulan is carried out in step b) at C₃ of dextran and/or pullulan. It is preferred for this embodiment that the carboxymethylation of at least one OH-group of dextran or pullulan is carried out in step b) at C₄ of dextran and/or pullulan.

In a further embodiment of the method of preparing a biocompatible hydrogel, in step c) the first polysaccharide is functionalized with 0.01-1.5 of A per monomeric repeating unit of the first polysaccharide. In a further preferred embodiment of the method of preparing a biocompatible hydrogel, in step c) the first polysaccharide is functionalized with 0.05-1.5 of A per monomeric repeating unit of the first polysaccharide. In a further preferred embodiment of the method of preparing a biocompatible hydrogel, in step c) the first polysaccharide is functionalized with 0.05-1.0 of A per monomeric repeating unit of the first polysaccharide.

For the method of preparing a biocompatible hydrogel, it is preferred that in step c) the second polysaccharide is functionalized with 0.01-1.5 of A′ per monomeric repeating unit of the second polysaccharide. In a further preferred embodiment of the method of preparing a biocompatible hydrogel, in step c) the second polysaccharide is functionalized with 0.05-1.5 of A′ per monomeric repeating unit of the second polysaccharide. In a further preferred embodiment of the method of preparing a biocompatible hydrogel according to the present invention, in step c) the second polysaccharide is functionalized with 0.05-1.0 of A′ per monomeric repeating unit of the second polysaccharide.

In a further embodiment of the method of preparing a biocompatible hydrogel, in step c) the first polysaccharide is functionalized with 0.01-1.5 of X per monomeric repeating unit of the first polysaccharide. In a further preferred embodiment of the method of preparing a biocompatible hydrogel according to the present invention, in step c) the first polysaccharide is functionalized with 0.05-1.5 of X per monomeric repeating unit of the first polysaccharide. In a further preferred embodiment of the method of preparing a biocompatible hydrogel according to the present invention, in step c) the first polysaccharide is functionalized with 0.05-1.0 of X per monomeric repeating unit of the first polysaccharide.

It is preferred that in step c) the second polysaccharide is functionalized with 0.01-1.5 of Y per monomeric repeating unit of the second polysaccharide. In a further preferred embodiment of the method of preparing a biocompatible hydrogel, in step c) the second polysaccharide is functionalized with 0.05-1.5 of Y per monomeric repeating unit of the second polysaccharide. In a further preferred embodiment of the method of preparing a biocompatible hydrogel, in step c) the second polysaccharide is functionalized with 0.05-1.0 of Y per monomeric repeating unit of the second polysaccharide.

In one embodiment of the method of preparing a biocompatible hydrogel, d is 1.

It is further preferred for the method of preparing a biocompatible hydrogel, that step e) is a thermo-induced cycloaddition reaction between X and Y for forming a crosslinked polymer.

In one embodiment of the method of preparing a biocompatible hydrogel, the content of N₃ is 0.01-1.5 N₃ per monomeric repeating unit of the first polysaccharide. In one preferred embodiment of the method of preparing a biocompatible hydrogel, the content of N₃ is 0.01-1.0 N₃ per monomeric repeating unit of the first polysaccharide. In one further preferred embodiment of the method of preparing a biocompatible hydrogel, the content of N₃ is 0.1-1.0 N₃ per monomeric repeating unit of the first polysaccharide.

In one embodiment of the method of preparing a biocompatible hydrogel, the content of Q is 0.01-1.5 per monomeric repeating unit of the second polysaccharide. In one preferred embodiment of the method of preparing a biocompatible hydrogel, the content of Q is 0.01-1.0 per monomeric repeating unit of the second polysaccharide. In one preferred embodiment of the method of preparing a biocompatible hydrogel, the content of Q is 0.1-1.0 per monomeric repeating unit of the second polysaccharide.

In one preferred embodiment of the method of preparing a biocompatible hydrogel, in step c) -A-X is linked to at least one primary or secondary OH-group of the first polysaccharide, preferably via at least one of C₂, C₃, C₄ or C₆ of the monomeric repeating unit of the first polysaccharide, more preferably via C₆ of the monomeric repeating unit of the first polysaccharide.

In one preferred embodiment of the method of preparing a biocompatible hydrogel, wherein in step c) -A′-Y is linked to at least one primary or secondary OH-group of the second polysaccharide, preferably via at least one of C₂, C₃, C₄ or C₆ of the monomeric repeating unit of the second polysaccharide, more preferably via C₆ of the monomeric repeating unit of the second polysaccharide.

It is further preferred for the method of preparing a biocompatible hydrogel, that the method is without the use of toxic reagents, preferably without the use of glutaraldehyde.

In one embodiment of the method of preparing a biocompatible hydrogel, the molecular weight of the unfunctionalized first polysaccharide is in the range from 5 to 2000 kDa. In one embodiment of the method of preparing a biocompatible hydrogel, the molecular weight of the functionalized first polysaccharide is in the range from 5 to 2500 kDa. In one preferred embodiment of the method of preparing a biocompatible hydrogel, the molecular weight of the functionalized first polysaccharide is in the range from 5 to 2000 kDa. In one more preferred embodiment of the method of preparing a biocompatible hydrogel, the molecular weight of the functionalized first polysaccharide is in the range from 5 to 1500 kDa. In one even more preferred embodiment of the method of preparing a biocompatible hydrogel, the molecular weight of the functionalized first polysaccharide is in the range from 10 to 1500 kDa.

In one embodiment of the method of preparing a biocompatible hydrogel, the molecular weight of the unfunctionalized second polysaccharide is in the range from 5 to 2000 kDa. In one embodiment of the method of preparing a biocompatible hydrogel, the molecular weight of the functionalized second polysaccharide is in the range from 5 to 2500 kDa. In one preferred embodiment of the method of preparing a biocompatible hydrogel, the molecular weight of the functionalized second polysaccharide is in the range from 5 to 2000 kDa. In one more preferred embodiment of the method of preparing a biocompatible hydrogel, the molecular weight of the functionalized second polysaccharide is in the range from 5 to 1500 kDa. In one even more preferred embodiment of the method of preparing a biocompatible hydrogel, the molecular weight of the functionalized second polysaccharide is in the range from 10 to 1500 kDa.

The hydrogel is obtainable by any method of preparing a biocompatible hydrogel as described above. It is preferred that said hydrogel comprises one or more encapsulated enzyme(s). It is further preferred that the hydrogel is a swellable or swollen hydrogel matrix.

A number of embodiments of the disclosure have been described. Nevertheless, it will be understood that various modifications may be made without departing from the spirit and scope of the present disclosure. Accordingly, other embodiments are within the scope of the following claims.

The examples set forth above are provided to those of ordinary skill in the art as a complete disclosure and description of how to make and use the embodiments of the disclosure, and are not intended to limit the scope of what the inventor/inventors regard as their disclosure.

Modifications of the above-described modes for carrying out the methods and systems herein disclosed that are obvious to persons of skill in the art are intended to be within the scope of the following claims. All patents and publications mentioned in the specification are indicative of the levels of skill of those skilled in the art to which the disclosure pertains. All references cited in this disclosure are incorporated by reference to the same extent as if each reference had been incorporated by reference in its entirety individually.

It is to be understood that the disclosure is not limited to particular methods or systems, which can, of course, vary. It is also to be understood that the terminology used herein is for the purpose of describing particular embodiments only and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains. The terms “over” and “under” are understood to show relative positions to other elements and not absolute positions with reference to the ground.

1. A method of fabricating a biosensor, the method comprising:
providing a wafer or other solid material with an electrode on a surface of the wafer or other solid material;
placing a drop of hydrogel on the electrode;
spinning the wafer or other solid material while the wafer is subjected to a partial vacuum; and
heating the wafer or other solid material.

2. The method of claim 1, wherein the hydrogel consists of pullulan, dextran, alginate, hyaluronic acid or mixtures thereof.

3. The method of claim 1, wherein the hydrogel consists of pullulan.

4. The method of claim 1, wherein the electrode is platinum, gold, graphite, or titanium or nanoparticles.

5. The method of claim 1, wherein the spinning has a maximum speed of up to 10000 rpm.

6. The method of claim 1, wherein spinning goes from a lower speed for a first interval of time to a higher speed at a second interval of time.

7. The method of claim 6, wherein the lower speed is less than 1000 rpm and the higher speed is greater than 1000 rpm.

8. The method of claim 1, wherein the heating the wafer comprises heating the wafer to at least 37° C. for over one hour.

9. The method of claim 1, wherein the hydrogel comprises one or more immobilized enzyme(s).

10. The method of claim 1, further comprising depositing a further hydrogel layer, not containing an enzyme.

11. The method of claim 1, wherein the hydrogel layer contains enzymes from the oxidase family.

12. The method of claim 11, wherein the enzymes from the oxidase family comprise at least one of glucose oxidase, lactate oxidase, uricase oxidase, alcohol oxidase, cortisol oxidase, xanthine oxidase, cholesterol oxidase, sarcosine oxidase.

13. The method of claim 10, further comprising depositing a metal layer on the further hydrogel layer.

14. The method of claim 1, further comprising depositing a metal layer on the hydrogel after the heating the wafer.

15. The method of any of claim 1, wherein the drop is in the range of 2 μL to 100 μL.

16. The method of any of claim 1, wherein the drop is over 100 μL.

17. A biosensor, comprising:
an electrode comprising a conductive layer over a substrate layer; and
a hydrogel layer over the conductive layer, the hydrogel layer being less than 3 micrometer thick and comprising enzymes immobilized within the hydrogel layer.

18. The biosensor of claim 17, further comprising a diffusion barrier over the hydrogel layer.

19. The biosensor of claim 18, wherein the diffusion barrier comprises metal.

20. The biosensor of claim 18, wherein the diffusion barrier comprises hydrogel without immobilized enzymes.

21. The biosensor of claim 20, further comprising a second diffusion barrier over the diffusion barrier, the second diffusion barrier comprising metal.

22. The biosensor of claim 17, wherein the hydrogel layer is 200 to 600 nm thick.

23. The biosensor of claim 19, wherein the diffusion barrier is 50 to 100 nm thick.
